(19) United States
(12) Patent Application Publication
(10) Pub. No.: US 2010/0292199 A1
(43) Pub. Date: Nov. 18, 2010

(54) TRANSCUTANEOUS PHARMACEUTICAL COMPOSITIONS CONTAINING A STEROID HORMONE

(76) Inventors: Elie Leverd, Castres (FR); Joël Bougaret, Francarville (FR)

Correspondence Address: BIRCH STEWART KOLASCH & BIRCH, PO BOX 747, FALLS CHURCH, VA, 22040-0747, US

(21) Appl. No.: 12/812,727

(22) PCT Filed: Dec. 4, 2008
(86) PCT No.: PCT/EP08/66808

§ 371 Date: Jul. 13, 2010

(30) Foreign Application Priority Data
Dec. 14, 2007 (FR) 0759864

(51) Int. Cl. A61K 31/56

(52) U.S. Cl. 514/178

(57) ABSTRACT

The present invention concerns aqueous-alcoholic, single-phase transcutaneous pharmaceutical compositions with an amount of alcohol of greater than 30% containing a steroid hormone combined with at least one penetrating agent selected from propylene glycol fatty acid esters, terpene derivatives, and mixtures thereof.

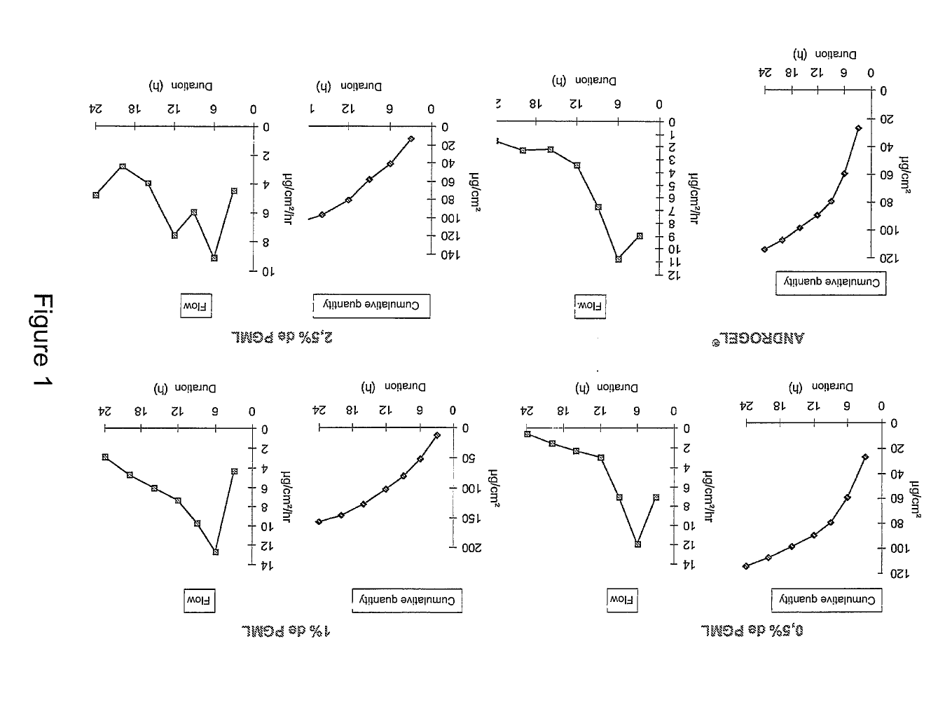

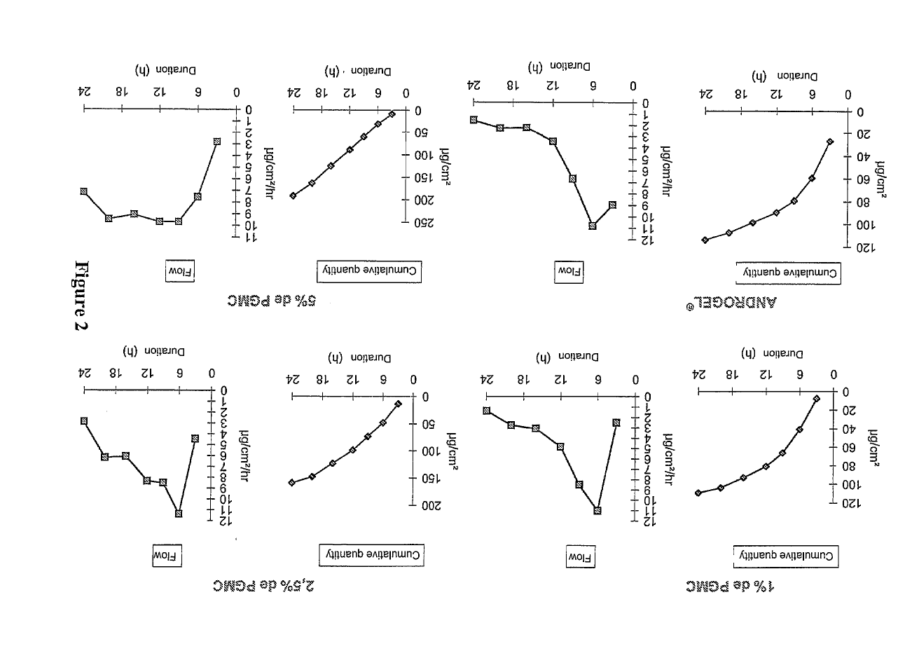

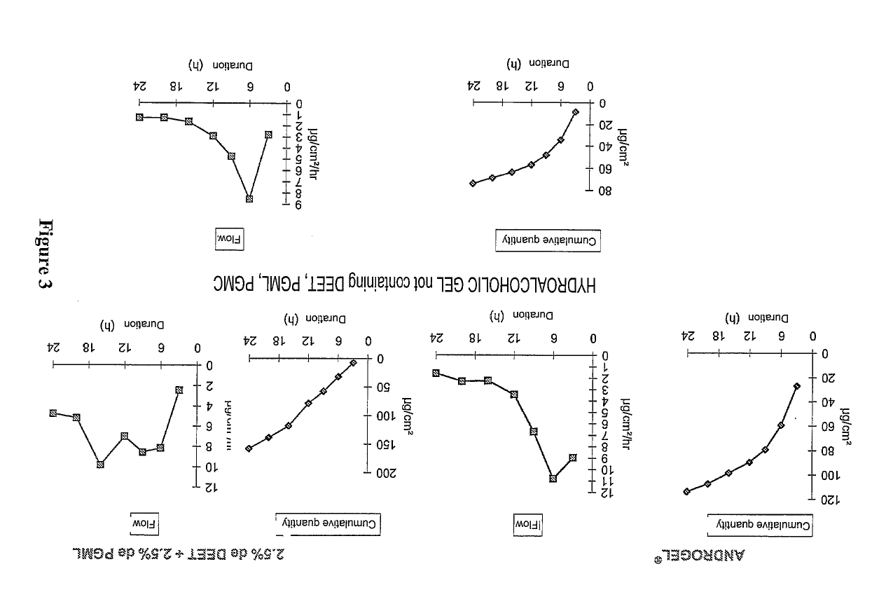

The present invention relates to the transcutaneous pharmaceutical compositions of hydroalcoholic type with a quantity of alcohol greater than 30% including a steroidal hormone.

Formulation of topical compositions applied to the skin involves taking into consideration the site of activity which the active ingredient must attain to exert its therapeutic activity. As a function of the degree of penetration of the molecule through the cutaneous barrier it is possible to distinguish:

local treatments,

local regional treatments,

systemic treatments with the active entity passing into the general blood circulation.

For the latter, topical compositions are qualified as transdermal systems. The galenic forms used most frequently are matricial patches or gel reservoirs. Their essential advantage is preventing degradation reactions which occur in the gastro-intestinal tract or during initial passage through the liver in the case of oral administration.

The development of transdermal patches originated in the 1970s and ended in agreement of the first patch by the FDA in 1979, which granted scopolamine for treating motion sickness. This development was continued and commercially available patches now contain active ingredients such as fentanyl, lidocaine, nicotine, nitroglycerine, oestradiol, testosterone, etc.

Patches definitely present advantages especially in terms of precision of the dose delivered, but also a certain number of disadvantages now identified as:

cutaneous irritation,

high industrial production cost,

limited possibility for incorporating large quantities of solvents and penetration agents, especially in matricial patches.

On the contrary, gels offer real advantages with respect to their cutaneous tolerance, their cosmetic appearance, their ease of preparation, their low industrial production cost and because they offer more extensive possibilities of active ingredient content, solvent and penetration agent due to a hydroalcoholic base.

For transdermal systems to be effective, it is necessary for the permeability of the skin to be substantial. This permeability is a function of a number of factors and depends on intrinsic characteristics of the active ingredient (Kp, Mw . . . ) and those of the excipients of the composition.

Among excipients, the presence of penetration agents modifies the properties of the barrier function of the skin, enabling better penetration of molecules.

The penetration agents are divided into several classes including, by way of example, fatty acids (example: oleic acid), terpenes (example: limonene), surfactants (example: polysorbates) or are represented by novel chemical entities (example: lauracapram or Azone).

A recent American publication (Pharmaceutical development and clinical effectiveness of a novel gel technology for transdermal drug delivery—Expert opinion. Ingo Alberti, Arnaud Grenier, Holger Kraus & Dario Norberto Carrara—Expert Opin. Drug Delivery (2005) 2(5) p935-950) analyses the booming market of transdermal gels. Beyond already marketed pharmaceutical specialties, many compositions are in clinic experimental phase and are the subject of intense developmental work.

Transdermal gels form the basis of numerous patents or public works and are often hydroalcoholic in nature with short-chain alcohols C¹-C⁴ and principally utilise derivatives of polyacrylic acid as gelling agents, such as carbomers or cellulosic derivatives such as cellulose ethers or gums such as gum Arabic or derivatives of polyvinylpyrrolidone or copolymers of polyoxyethylene and polyoxypropylene (see WO2006/125642 by ANTARES PHARMA).

Their composition includes penetration agents of varied type (see WO2002/17926 by Laboratoires BESINS ISCOVESCO and UNIMED PHARMACEUTICALS and WO2002/11768 by ANTARES PHARMA).

The underlying problem with compositions of the prior art is that even though they allow satisfactory transdermal flow of active ingredient, this flow is unstable over time following application. In fact, a flow peak is noticed in the few hours following application, though this flow drops rapidly thereafter.

Now, it turns out to be necessary to provide a composition which can effectively allow high transdermal flow which is kept highly regular over time following application, similar to what occurs with a patch.

The aim of the present invention is therefore transcutaneous pharmaceutical compositions of monophasic hydroalcoholic type with a quantity of alcohol greater than 30% containing a steroidal hormone linked to at least one fatty acid ester and propylene glycol as penetration agent.

The hydroalcoholic phase is constituted by a mixture of water and short-chain alcohols C¹-C⁴ and more precisely by a mixture of water and ethylic or isopropylic alcohol in proportions varying from 80/20 to 20/80 (m/m), preferably in a fork of 70/30 to 30/70. The quantity of alcohol present in the hydroalcoholic phase is greater than 30%. In fact, a quantity of alcohol greater than 30% allows good solubilisation of steroidal hormones.

These novel pharmaceutical compositions topical can also contain an apolar solvent soluble such a s N,N-di-ethyl-m-toluamide or DEET in the hydroalcoholic phase, with a concentration varying from 0.5% to 10% (m/m) of the composition and preferably varying from 0.1% to 0.5% (m/m) of the composition. By way of non-limiting example, these apolar solvents are represented by N,N-di-ethyl-m-toluamide or DEET.

These novel compositions are more precisely gels or hydroalcoholic solutions with a quantity of alcohol greater than 30%. In a controlled manner they administer solubilised steroidal hormones such as by way of non-limiting example testosterone, oestradiol, progesterone or their derivatives, transcutaneously, systemically. In the case of a gel form the composition according to the present invention could comprise at least one gelling agent traditionally used in the pharmaceutical industry, such as carbomer, for example.

According to another characteristic of the invention, the composition contains at least one fatty acid ester and propylene glycol as penetration agent with a concentration varying from 0.5% to 10% (m/m) of the composition.

According to another characteristic of the invention, the composition contains 2.5% (m/m) of propylene glycol monolaurate.

According to another characteristic of the invention, the composition contains 5% (m/m) propylene glycol monocaprylate.

According to another characteristic of the invention, the composition contains a mixture, preferably equiweighted, of propylene glycol monolaurate and propylene glycol monocaprylate.

The compositions according to the present invention can be in any form adapted to topical application, such as for example a solution, a spray or even serve in a transdermal device of reservoir patch type.

Also, the compositions according to the present invention could comprise any dermatologically acceptable excipient such as thickeners, dyes, aromas or perfume or even an emollient to counterbalance the desiccating effect of the alcoholic compound. Such emollients could be selected from glycerol or propylene glycol.

The concentration of steroidal hormone of these gels or solutions varies from 0.1% to 5% (m/m) of the composition. The concentration of penetration agent of fatty acid ester and glycol or terpenic derivative type varies from 0.5% to 10% (m/m) of the composition.

The following examples illustrate the invention.

Testosterone is a molecule of low molecular mass (MM: 288), of hydrophobic nature as indicated by its octanol/water partition coefficient (log P=3.3), insoluble in water and soluble in ethyl alcohol.

BESINS laboratories market ANDROGEL(R), a hydroalcoholic gel [degree of alcohol close to 71% (m/m)], dosed at 1% (m/m) of testosterone, whereof the excipient composition comprises, apart from water and ethanol, 0.5% (m/m) of isopropyl myristate as penetration agent and carbomer as gelling agent.

Hydroalcoholic gels [degree of alcohol: 71% (m/m)] were prepared, based on carbomer, with 1% (m/m) of testosterone, incorporating growing quantities of propylene glycol monolaurate (PGML): 0.5%, 1% and 2.5% (m/m) of propylene glycol monocaprylate (PGMC): 1%, 2.5% and 5% (m/m) or the mixture 2.5% (m/m) DEET+2.5% (m/m) PGML or 2.5% (m/m) of levomenthol.

The formulae are indexed in the table below.

| Compound name | Formulae (g) | | | | | | | |
|---|---|---|---|---|---|---|---|---|
| Testosterone | 1 | 1 | 1 | 1 | 1 | 1 | 1 | 1 |
| DEET | | | | | | | 2.50 | |
| Propylene glycol | 0.50 | 1 | 2.50 | | | | 2.50 | |
| monolaurate⁽¹⁾ | | | | | | | | |
| Propylene glycol | | | | 1 | 2.50 | 5 | | |
| monocaprylate⁽²⁾ | | | | | | | | |
| Levomenthol | | | | | | | | 2.50 |
| Ethanol 96 | 71 | 71 | 71 | 71 | 71 | 71 | 71 | 71 |
| Carbomer⁽³⁾ | 0.60 | 0.60 | 0.60 | 0.60 | 0.60 | 0.60 | 0.60 | 0.60 |
| Triethanolamine | 0.17 | 0.18 | 0.18 | 0.18 | 0.18 | 0.18 | 0.18 | 0.18 |
| Purified water sqf | 100 | 100 | 100 | 100 | 100 | 100 | 100 | 100 |

⁽¹⁾Lauroglycol 90 ® - Supplier: Gattefosse
⁽²⁾Capryol 90 ®) - Supplier: Gattefosse
⁽³⁾Carbopol 980NF ® - Supplier: Noveon

The novel transcutaneous pharmaceutical compositions forming the subject matter of the invention are prepared according to a production process entailing stages of dispersion, dissolution and mixing, well known to the expert.

The production process is exemplified below, for preparation of 100 g of gel containing 2.5% (m/m) of PGML.

dissolve 1 g of testosterone in 71 g ethanol 96% using a bar magnet, with magnetic stirring,

add and fully dissolve 2.5 g of propylene glycol monolaurate (Lauroglycol 90®, supplier: Gattefosse),

disperse 0.6 g of carbomer (Carbopol 980NF®, supplier: Noveon),

with mechanical stirring (three-blade propeller), slowly add 24.72 g of purified water then 0.18 g of triethanolamine,

homogenise.

Freeing and diffusion of the testosterone were apprehended by an ex-vivo permeation test on skin of mice in Franz cell.

Experimental conditions are the following:

dorsal skin of “swiss nu” mice,

cell volume: 22.5 ml,

membrane surface: 4.95 cm²,

composition of the receptor compartment:

water containing NaCl: 0.9%, polyoxyethylene 20 oleyl ether: 1% and NaN₃: 0.1%

The results of passage of the testosterone are presented in the attached figures and are expressed in cumulative quantity per surface unit (μg/cm²) and at speed (flow) expressed in μg/cm²/h., as a function of time over 24 h.

Thanks to the compositions according to the present invention it is possible to modulate and process the profile of the transcutaneous passage of testosterone by varying the quality and quantity of the penetration agent of fatty acid ester and glycol type incorporated in the hydroalcoholic gel. This possibility adapts a galenic gel or hydroalcoholic solution form at a given therapeutic activity (notion of transcutaneous chronotherapy), since either a peak effect or more interestingly passage with a linear kinetics (zero order) will be attained, for example for a concentration of propylene glycol monolaurate of 2.5% (m/m) or propylene glycol monocaprylate of 5% (m/m). Adding an apolar solvent such as DEET to a composition according to the present invention containing propylene glycol monolaurate as permeation agent does not modify the liberation and diffusion profile of testosterone and also produces a quasi constant permeation flow.

In summary, rapid absorption of testosterone in the first hours is followed by a flow which [missing word: probably increases] or even decreases or is maintained at a value near 10 μg/cm²/h in the best case. This is perfectly controlled by the increase in propylene glycol monolaurate or monocaprylate content, but also by incorporation of association such as DEET+propylene glycol monolaurate [2.5%+2.5% (m/m)] where the flow is linearised over close to 24 h which is outstanding and completely unexpected.

Apart from testosterone, the presence of an apolar solvent favours solubilisation of a lipophilic or amphiphilic entity in the hydroalcoholic formulation.

It is evident to the expert that hydroalcoholic transcutaneous pharmaceutical compositions forming the subject matter of the invention may also contain other common ingredients for this type of formulation, such as emollients (glycerol, propylene glycol), preservatives (antimicrobial and antioxidant), dyes, perfumes, etc. involved in imparting cosmetic qualities to said preparation.

The compositions according to the present invention are intended for topical application within the scope of hormonal substitution treatment both in men and in women. The compositions according to the present invention are adapted particularly to substitute hormone treatment in women in light of complementation of testosterone in women presenting conditions associated with menopause. In fact, relative to transdermal devices of patch type, the compositions according to the present invention are adapted particularly to administration of a small quantity of testosterone on a small cutaneous surface. Also, using a topical composition of gel or spray type offers usage comfort and a considerable aesthetic advantage for patients.

1. Transcutaneous pharmaceutical compositions of monophasic hydroalcoholic type with a quantity of alcohol greater than 30% and containing a steroidal hormone linked to at least one fatty acid ester and propylene glycol as penetration agent.

2. The transcutaneous pharmaceutical compositions as claimed in claim 1, characterised in that they contain N, N-di-ethyl-m-toluamide or DEET as apolar solvent soluble in the hydroalcoholic phase, with a concentration varying from 0.1 to 0.5% (m/m) of the composition.

3. The transcutaneous pharmaceutical compositions as claimed in any one of claims 1 and 2, characterised in that they are in the form of gels or solutions containing testosterone as steroidal hormone, with a concentration varying from 0.1% to 5% (m/m) of the composition.

4. The transcutaneous pharmaceutical compositions as claimed in any one of claims 1 to 3, characterised in that they contain at least one gelling agent, such as a carbomer.

5. The transcutaneous pharmaceutical compositions as claimed in any one of claims 1 to 4, characterised in that the concentration of the fatty acid ester and propylene glycol varies from 0.5% to 10% (m/m) of the composition.

6. The transcutaneous pharmaceutical compositions as claimed in any one of claims 1 to 5, characterised in that they contain 2.5% (m/m) of propylene glycol monolaurate.

7. The transcutaneous pharmaceutical compositions as claimed in any one of claims 1 to 6, characterised in that they contain 5% (m/m) of propylene glycol monocaprylate.

8. The transcutaneous pharmaceutical compositions as claimed in any one of claims 1 to 7, characterised in that they contain a mixture, preferably equiweighted, of propylene glycol monolaurate and propylene glycol monocaprylate.

9. The transcutaneous pharmaceutical compositions as claimed in any one of claims 1 to 8, characterised in that they contain a terpenic derivative as additional penetration agent with a concentration varying from 0.5% to 10% (m/m) of the composition.

10. The transcutaneous pharmaceutical compositions as claimed in claim 9, characterised in that they contain 2.5% (m/m) of levomenthol.
